(19) United States
(12) Patent Application Publication
(10) Pub. No.: US 2014/0335027 A1
(43) Pub. Date: Nov. 13, 2014

(54) HEATINESS AND SALIVARY SECRETORY IMMUNOGLOBULIN

(71) Applicants: David Lungpao Chang, Las Vegas, NV (US); Zheng Yang, Guangzhou (CN); ZhaoSheng Huang, Guangzhou (CN); SiJun Liu, Guangzhou (CN)

(72) Inventors: David Lungpao Chang, Las Vegas, NV (US); Zheng Yang, Guangzhou (CN); ZhaoSheng Huang, Guangzhou (CN); SiJun Liu, Guangzhou (CN)

(73) Assignee: Colgate-Palmolive Company, New York, NY (US)

(21) Appl. No.: 14/365,713

(22) PCT Filed: Dec. 21, 2011
(86) PCT No.: PCT/CN2011/002148

§ 371 Date: Jun. 16, 2014

(51) Int. Cl. G01N 33/53; A61K 31/085; G06F 19/00; A61K 33/42; A61K 33/30; A61K 31/194; A61K 9/00; A61K 33/16

(52) U.S. Cl. CPC G01N 33/53; A61K 9/006; A61K 31/085; A61K 33/16; A61K 33/42; A61K 33/30; A61K 31/194; G06F 19/34
USPC 424/52; 436/501; 424/49; 702/19

(57) ABSTRACT

Described herein are methods of assessing the severity of heatiness in a patient, comprising measuring salivary S-IgA levels, which are found to correlate negatively with the degree of heatiness. The invention further provides, i.a., diagnostic kits, methods of treatment, and the use of oral care compositions to treat heatiness.

### BACKGROUND

“Heatiness” is a term used to describe symptoms associated with excessive “internal heat” in our body, which form a syndrome recognized in Traditional Chinese Medicine (TCM). Heatiness is characterized by dryness of mouth, redness, swelling, heat, and pain. Different types of heatiness are recognized, for example, sthenia fire, e.g., characterized by red face/eyes/tongue, and rapid strong pulse, and deficient fire, e.g., sleepless, dry mouth, nosebleed, and rapid weak pulse. Fundamentally, heatiness is considered to be a maladjustment of the balance of yin-yang required to maintain health. The goal of medication under TCM principles is to adjust the body's balance and restore health.

Under TCM principles, heatiness is viewed as the clinical manifestation of exogenous evils transforming into heat or internal depression turning into fire. It exhibits somewhat different symptoms in different parts of the body. The usual symptoms of heatiness within the mouth for example are as follows: boils of tongue and lips, bitter taste in mouth and bad breath, dry mouth and cheilosis, orolingual sores, swelling and reddish of gingiva, toothache and bleeding gum, red tongue with yellowish furry coating, reddish margin of tongue without furry coating. These local symptoms overlap with symptoms of diseases such as gingivitis, periodontitis, acute coccus infective stomatitis, herpetic stomatitis, lingual papillitis, acute parotitis and so on, which are described in Western medicine, but under TCM teachings, the mind and body are viewed as an organic whole, so that the local symptoms in the mouth cannot be effectively disassociated and treated separately from the underlying systemic imbalance resulting in heatiness. A simple one-to-one correlation between diseases recognized by Western medicine and heatiness conditions is thus very difficult. The categorization of disease states is different from Western medicine, and treatment of heatiness typically involves particular diets and/or use of traditional Chinese herbal medicines.

There is a long history of developing understanding and treatments for heatiness, and despite poor acceptance and understanding of TCM principles in Western medicine, millions can testify as to the efficacy of TCM approaches. Existing TCM approaches, including heatiness diagnosis, are nevertheless based largely on personal experience, subjective opinion, and anecdotal reports. There is a lack of symptomalogical observation and semi-quantitative or quantitative diagnostic standards, as well as a lack of biomarkers or other objective and consistent physical criteria, that would allow a more rigorous and consistent study of heatiness and its treatment, and also allow a better synthesis and understanding of the relationship between diseases as understood and treated under Western medical principles, and heatiness as understood and treated under TCM principles.

In Western medicine, there is some evidence that oral health may affect or be affected by systemic health, including cardiovascular health, that systemic disorders may increase the risk of some oral diseases, and that the oral tissues may serve as a gateway for systemic infection. The oral cavity is protected from infection by the mucosal immune system, which operates to prevent invasion of the mucosa by potentially pathogenic microbes and to prevent the uptake of antigens. Despite the importance to an individual of mucosal immunity, as the first line of defense against most pathogens and antigens, it is one of the least understood areas of human immunology. Activated B cells in the mucosal epithelia produce a dimeric form of immunoglobulin A (IgA) joined by a J-chain and linked to a secretory component (SC). The secretory component facilitates secretion of the complex from the mucous membranes. This secretory IgA (S-IgA) is the main immunoglobulin found in mucous secretions, including saliva.

### SUMMARY

We have surprisingly discovered that the level of S-IgA in saliva negatively correlates with the degree of heatiness in patients. In general, when a person has heatiness, the S-IgA value in saliva is low compared to a normal control.

We conducted a clinical study, including patients diagnosed using TCM principles as suffering from heatiness and normal patients, to document symptoms of heatiness systematically and objectively. Based on this study, we developed a scoring system, giving different symptoms different weight, and identified a critical diagnostic score as corresponding to patients suffering from heatiness. The patients could be further categorized as to the severity of their condition and the type of heatiness on the basis of these scores. In the course of this study, we also measured salivary flow rate and levels of salivary lysozyme, salivary amylase, and salivary S-IgA in the patients.

We found that there is a significant negative correlation between the level of S-IgA in saliva and the severity of heatiness. This was surprising, because there was no significant correlation with the other parameters measured in the saliva analysis. Accordingly, salivary S-IgA levels can be used as a clinical parameter to diagnose heatiness and to assess the effectiveness of anti-heatiness treatments.

We found that S-IgA increase scope is significant correlated to the scope of diagnostic score reduction among heatiness cases.

We found that combination use of diagnostic score table and S-IgA to diagnosis heatiness can raise the concordance rate vs clinicians' results.

The invention thus provides, in one embodiment, a method of raising heatiness diagnosis concordance rate, quantitatively measuring the effect of anti-heatiness treatment, or diagnosing, assessing or monitoring heatiness comprising measuring S-IgA levels in saliva, wherein a lower level of S-IgA as compared to a normal or baseline level corresponds to heatiness or a worsening of a heatiness condition respectively, and S-IgA used for diagnosis together with diagnostic score table can raise diagnosis concordance rate; as well as a kit for use in such a method, comprising means for measuring S-IgA levels in saliva, for example using antibodies to S-IgA.

In another embodiment, the invention provides a method of treating heatiness in patients so diagnosed, comprising administering an oral care product, e.g., a toothpaste or mouthrinse, comprising cooling agents, particularly herbs from TCM and/or antibacterial agents such as triclosan or a zinc salt or oxide, e.g., zinc oxide, zinc citrate, or zinc lactate, together with the use of such products for such treatment.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

In a first embodiment, the invention provides a method (Method 1) of raising heatiness diagnosis concordance rate, quantitatively measuring the effect of anti-heatiness treatment, diagnosing, assessing or monitoring heatiness in a patient comprising measuring salivary S-IgA in a sample of saliva from the patient.

For example, the invention provides
1.1. Method 1 wherein a lower level of salivary S-IgA correlates negatively with the severity of heatiness.
1.2. Method 1 or 1.1 wherein a patient is also assessed for symptoms in accordance with the following Table A (below).

TABLE A

| Symptoms | value | Symptoms | value | Symptoms | value |
|---|---|---|---|---|---|
| Mouth ulcer | ***23 | Ocular | *8 | Low-grade | **15 |
| | | itching | | fever | |
| Dry mouth | **14 | Secretion of | **18 | Insomnia | *8 |
| | | the eye | | | |
| Bitter taste | **15 | Tinnitus | **11 | Tantrum | **17 |
| of mouth | | (ear ringing) | | | |
| Halitosis | **17 | Dry pharynx | **13 | Yellow urine | *9 |
| Gum swelling, | **17 | Sore throat | **15 | Constipation | *8 |
| pain or | | | | | |
| bleeding | | | | | |
| Red tongue | *9 | Acne | *6 | Stuffiness | *6 |
| Dryness in the | **15 | Dizziness | *6 | Yellow musci | **15 |
| nasal cavity | | | | | |
| Nose bleeding | *10 | Scurf | *6 | Frequent and | **11 |
| | | (scales, flakes) | | weak pulse | |
| Dryness of | *9 | Dryness-heat | **17 | Frequent and | **13 |
| the eyes | | | | strong pulse | |

1.3. The preceding method wherein the symptoms assessed are given a weight approximately corresponding to or approximately proportional to the numerical values listed in Table A.
1.4. The preceding method wherein a diagnosis of heatiness is given to a patient having (i) a relatively low (less than normal or baseline) value for salivary S-IgA and (ii) a score of 60 or higher, e.g., at least 63, using the numerical values in Table A, or a value proportional to or corresponding to that score.
1.5. Any of the foregoing methods wherein the degree of heatiness is categorized based on severity, e.g. common, serious, or very serious, e.g., using the numerical values in Table A where common heatiness corresponds to a score of 63 . . . 120; serious heatiness corresponds to a score of 121 . . . 150; and very serious heatiness corresponds to a score of >150.
1.6. Any of the foregoing methods wherein the level of S-IgA is measured using an antibody to S-IgA, e.g., to the secretory component of the S-IgA.
1.7. Any of the foregoing methods wherein the patient has not had any oral intake for at least one hour prior to the measurement of the salivary S-IgA.
1.8. Any of the foregoing methods wherein the normal or baseline level of S-IgA is calculated taking into consideration the season of the year.
1.9. Any of the foregoing methods 1 wherein S-IgA is used as an auxiliary diagnosis index to raise concordance rate vs clinicians in terms of heatiness diagnosis.
1.10. Any of the foregoing methods wherein S-IgA is used as an auxiliary assessment index to quantitatively measure the effect of anti-heatiness treatment together with diagnostic score table which is a main assessment index

In another embodiment, the invention provides a method to classify types of heatiness using a Principal Component Analysis (Prin), for example
2. A method (Method 2) of diagnosing a particular type of heatiness comprising assessing a patient for symptoms as set forth on Table B (below).

TABLE B

| Prin | Heatiness Type | Primary Heatiness Symptoms |
|---|---|---|
| Prin 1 | Sthenia Fire | bitter taste of mouth, halitosis, nasal obstruc- |
| | | tion, tinnitus, dizzy, scurf, dryness-heat, |
| | | tantrum, yellow urine, yellow musci, frequent |
| | | and strong pulse |
| Prin 2 | Deficient | dry mouth, halitosis, uloncus, dryness in the |
| | Fire | nasal cavity, sore throat, dizzy, dryness-heat, |
| | | low-grade fever, red tongue, frequent and |
| | | weak pulse |
| Prin 3 | Heart Fire | mouth ulcer, dryness of eye, sore-throat, |
| | | insomnia, yellow musci |
| Prin 4 | Lung-stomach | dry mouth, uloncus, tinnitus, dry pharynx, |
| | Sthenia | acne, constipation, yellow musci |
| Prin 5 | Hepatic and | uloncus, tinnitus, low-grade fever, tantrum, |
| | renal yin | constipation, red tongue, yellow musci, |
| | deficiency | frequent and weak pulse |
| | Fire | |
| Prin 6 | Asthenia of | nasal obstruction, acne, red tongue, yellow |
| | pulmonary yin | musci, frequent and weak pulse |

2.1. Method 2 wherein the patient receives a diagnosis of heatiness in accordance with any of Method 1 et seq, then is assessed under Method 2 to diagnose qualitatively the type of heatiness.
2.2. Method 2 or 2.1 wherein the patient is given a value for each principal component (Prin) in Table B, wherein the highest absolute value establishes the type of heatiness affecting the patient.

In another embodiment, the invention provides a machine readable program and a computer wherein the calculations to determine a weighted score for heatiness and/or type of heatiness in accordance with any of Methods 1, et seq. or Methods 2 et seq. are performed by the machine readable program and the computer based on input regarding the presence or absence of relevant symptoms, e.g., as set forth in Table A. For example, the invention provides a computer-assisted system for self-diagnosis or diagnosis by a dental practitioner, wherein a user enters data regarding the level of S-IgA and the presence or absence of symptoms as listed in Table A, e.g., via a website, and the data is uploaded into a calculating program, e.g., a spreadsheet program such as Microsoft Excel, to permit calculation of a heatiness diagnostic score, and the score is then displayed to the user. Optionally, based on the score, information regarding heatiness and appropriate methods of treatment is also provided to the user.

In another embodiment, the invention provides
3. A method of treatment (Method 3) comprising diagnosing a patient in accordance with any of Methods 1 or 2 as suffering from heatiness, and treating the patient by administering an oral care product, e.g., a toothpaste or a mouthwash, comprising an effective amount of an antiheatiness agent.
3.1. Method 3 wherein the antiheatiness agent is recognized in TCM, for example comprising one or more anti-heatiness agents selected from: berberine and jateorhizine from rhizome of Chinese goldthread, baicalin from root of Baikal skullcap, matrine and oxymatrine from root of lightyellow sophora, andrographolide from common andrographis herb, houttuynine sodium bisulfite from herb of heartleaf houttuynia, mangiferin from rhizome of anemarrhena, and alkanin from redroot gromwell; chrysanthemum lactone from flos chrysanthemi indici, chlorogenic acid from honeysuckle flower, tea polyphenols from tea leaf, and magnolol from bark of magnolia.
3.2. Method 3 or 3.1 wherein the antiheatiness agent comprises a synthetic antibacterial or anti-inflammatory agent.
3.3. Method 3.2 wherein the antiheatiness agent is selected from triclosan, zinc citrate, zinc lactate, zinc oxide and combinations thereof, e.g., comprising 0.1-1% triclosan and/or 1-3% zinc citrate;
3.4. Any of the foregoing methods wherein the oral care product is administered to the oral cavity of the patient on at least a daily basis for a period of at least one week or until symptoms subside.

In a further embodiment, the invention provides a kit for measuring, diagnosing or monitoring S-IgA, comprising antibody to S-IgA, e.g. to the secretory component (SC) of S-IgA, together with instructions for use.

In a further embodiment, the invention provides an oral care product, e.g., a toothpaste or a mouthwash, comprising an effective amount of an antiheatiness agent, for use in a method of treating heatiness, for example to treat a patient diagnosed in accordance with any of Method 1 et seq. or Method 2, et seq. e.g.,
wherein the antiheatiness agent is recognized in TCM, for example comprising one or more anti-heatiness agents selected from: berberine and jateorhizine from rhizome of Chinese goldthread, baicalin from root of Baikal skullcap, matrine and oxymatrine from root of lightyellow sophora, andrographolide from common andrographis herb, houttuynine sodium bisulfite from herb of heartleaf houttuynia, mangiferin from rhizome of anemarrhena, and alkanin from redroot gromwell; chrysanthemum lactone from flos chrysanthemi indici, chlorogenic acid from honeysuckle flower, tea polyphenols from tea leaf, and magnolol from bark of magnolia; and/or
wherein the antiheatiness agent comprises a synthetic antibacterial or anti-inflammatory agent, and/or
wherein the antiheatiness agent is selected from triclosan, zinc oxides or salts (zinc oxide, zinc citrate, and/or zinc lactate), and combinations thereof, e.g. comprising 0.1-1% triclosan and/or 1-3% zinc citrate;
   for example wherein the oral care product is selected from a dentifrice comprising 0.3% triclosan, 2% methyl vinyl ether/maleic anhydride copolymer and 0.32% sodium fluoride in a silica base and a dentifrice comprising 1.1% sodium monofluorophosphate, 1.5% methyl vinyl ether/maleic anhydride copolymer, 1.3% % pyrophosphate and 2% zinc citrate trihydrate in a silica base.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

### EXAMPLES

### Example 1

### Clinical Assessments and Determination of Statistical Correlation Between S-IgA and Heatiness

Experimental Materials:
Apparatus: SN-695B Radioimmunity Measuring Instrument, Rihuan No. 1 Instrument Factory, Shanghai Atomic Nucleus Research Institute; Olympus AU 5421, Japan; Ultra-Violet And Visible Spectrophotometer Model 752, Shanghai.
Reagents: S-IgA Kit, Beijing North BiotecHuology Research Institute, Batch No: (061120, 061230, 070412, 070420); α-Diastasimetry Kit, HUMAN Co, Germany, Batch No: H100; LZM Kit, Nanjing Jiancheng Bioengineering Research Institute, Batch No: (20060922, 20061025, 20070412)

121 heatiness cases are selected from the volunteers as meeting heatiness criteria, based on assessment of clinicians specializing in TCM. 27 symptoms of heatiness are identified as correlating statistically with the TCM assessment of heatiness, and weighted according to the degree of correlation with the TCM diagnosis, to provide a more objective criteria. The symptoms are weighted based on their relative contribution to the diagnosis of heatiness, in accordance with the chart below. Patients scoring 63 or higher are considered to be suffering from heatiness.

TABLE A

| Symptoms | value | Symptoms | value | Symptoms | value |
|---|---|---|---|---|---|
| Mouth ulcer | ***23 | Ocular | *8 | Low-grade | **15 |
| | | itching | | fever | |
| Dry mouth | **14 | Secretion of | **18 | Insomnia | *8 |
| | | the eye | | | |
| Bitter taste | **15 | Tinnitus | **11 | Tantrum | **17 |
| of mouth | | (ear ringing) | | | |
| Halitosis | **17 | Dry pharynx | **13 | Yellow urine | *9 |
| Gum swelling, | **17 | Sore throat | **15 | Constipation | *8 |
| pain or | | | | | |
| bleeding | | | | | |
| Red tongue | *9 | Acne | *6 | Stuffiness | *6 |
| Dryness in the | **15 | Dizziness | *6 | Yellow musci | **15 |
| nasal cavity | | | | | |
| Nose bleeding | *10 | Scurf | *6 | Frequent and | **11 |
| | | (scales, flakes) | | weak pulse | |
| Dryness of | *9 | Dryness-heat | **17 | Frequent and | **13 |
| the eyes | | | | strong pulse | |

Of the patients suffering from heatiness, those who recovered in a natural way are determined as the self-control group. Their follow-up data are collected. At the same time, 60 healthy cases are selected as the blank control group. Henan and Hunan researchers of the project strictly adopted the same methods of case screening, sampling and testing as in Guangzhou. In Hunan, 121 heatiness cases, 61 healthy cases and 70 self-control cases are selected while in Henan 143 heatiness cases, 74 healthy cases and 70 self-control cases are selected.

Saliva samples are taken from the healthy, heatiness, and naturally recovered cases at 9:00-11:00 am and 2:00-4:00 pm. After the mouth is rinsed with water, the subject is instructed to sit quietly for 3 min, holding the saliva naturally secreted, and then spat all saliva (unstimulated whole saliva) into a tube at one minute's interval. The saliva secreted during 10 minutes is collected and the salivary flow rate is calculated. If the total amount of saliva secreted during 10 minutes is less than 3 ml, the collection time could be lengthened till the amount reached 3 ml. Salivary flow rate (SFR) in ml/min=total amount of saliva collected during 10 min divided by 10 min. The collected saliva is divided into three lots, 1 ml each, to be kept at −20° C. Then salivary amylase (AMS), salivary lysozyme (LYZ) and secreted immunoglobulin (S-IgA) are measured.

AMS: After the specimen is diluted 20 times by physiological saline after freeing and thawing treatment, automatic sampling is performed with Olympus AU 5421 and then the reagent kit detection is conducted.

LYZ: Applying the ultra-violet and visible spectrophotometer model 752, the reagent kit detection is conducted. In a turbid bacterial solution of a certain concentration, LYZ hydrolyzes the polypeptide in the bacterial cell wall to bring about bacterial schizolysis so as to decrease the concentration while transmittancy increases. Thus, the LYZ content can be determined according to the change of the turbidity.

After being stirred evenly, the tubes of specimen undergo a water bath at 37° C. for 15 min. They were taken out immediately into iced water below 0° C. After water bath for 3 min, the specimens were poured, one tube after another, in the 1 cm photoelectrometric tubes. At 530 nm, transmittancy is adjusted with distilled water to 100° C. Colorimetry is performed to measure the transmittancy T15, which is the transmittancy after water bath at 37° C. for 15 min.

LYZ content (μg/ml)=(measured tube transmittancy UT15−blank tube transmittancy OT15/(transmittancy ST15−blank tube transmittancy OT15)*standard tube concentration*specimen dilution folds

S-IgA (μg/ml): SN-695B radio-immunity apparatus R is employed and reagent kit detection is conducted. S-IgA exists widely in blood and various kinds of exudates. As the main type of immunoprotein in the exudates, it is a mucosal specific defense factor. This kit employs double-antibody sandwich to assay S-IgA in serum and exudates. Employment of double-antibody is intended for the specific antibody of the secretory component portion (SC) in S-IgA. This method is of high specificity, sensitivity, and reliability. At first, the antibody wrapping the polystyrene nanosphere is combined with S-IgA of the specimen to form the immunity composite Antibody-S-IgA. With the addition of the labelled antibody 125I antibody, a three-layer sandwich Antibody-S-IgA-125I-Antibody is formed. Measure the 125Icpm values over the spheres and control count them with the standard curve or the standard value cpm. Then the S-IgA content in the specimen becomes known.

After the freezing and thawing treatment, the specimen is diluted to 1:1000 with physiological water. To avoid test errors, tests must be completed at one go. Before calculation, S0cpm should be deducted from each tube. The specimen concentration to be measured is treated with the computer software IRMA to get the measured value as the S-IgA concentration. Now it is multiplied by the dilute folds. The result is the concentration of the specimen.

Various factors are assessed for statistical correlation with these four indicies. There is a significant different in AMS between the two genders; the standard deviation, however, is large, which is possibly due to sampling, individual difference, and testing methods. As to the other three indices, there is no significant difference between the sexes. The indices of all four indices vary with seasons, reflecting the occurrence of diseases in the four seasons.

Self-Paired Comparison of the Changes of SFR, LYZ, AMS, and S-IgA in heatiness: By employing the random table, a follow-up study is implemented of 60 of the 121 heatiness cases. After the cases recover naturally, saliva is taken from them. The values of the various parameters are measured. The self control has an advantage of avoiding individual deviation. The figures of the self control undergo T test and SPSS13.0 statistical analysis. The results are as follows:

Self-PairedComparison of the Changes
of the Various Parameters ( X̅ ± S)

| Indices | State | N | X̅ ± S | T | P |
|---|---|---|---|---|---|
| SFR | Heaty | 60 | 0.43 ± 0.29 | −0.673 | 0.503 |
| | Healthy | 60 | 0.45 ± 0.23 | | |
| LYZ | Heaty | 60 | 33.42 ± 5.83 | 3.087 | 0.003 * |
| | Healthy | 60 | 34.25 ± 5.20 | | |
| AMS | Heaty | 60 | 112916 ± 83073 | 2.315 | 0.024 * |
| | Healthy | 60 | 87309 ± 86224 | | |
| S-IgA | Heaty | 60 | 439.77 ± 607.10 | −4.530 | 0.000 * |
| | Healthy | 60 | 1348.57 ± 1706.90 | | |

From the results of the study it is seen that SFR showed no difference before and post to recovery after heatiness. LYZ had a significant decline before recovery. AMS had a significant decline following natural recovery but S-IgA rose significantly post recovery, implying that S-IgA is probably a protective protein in the body.

Heatiness correlation with SFR, LYZ, AMS and S-IgA: According to the diagnostic score table, the score values of the cases are calculated. The rank-sum relativity test is performed of the values of SFR, LYZ, AMS and S-IgA, of the same individual cases, which are analyzed statistically to derive the correlation of the diagnostic score values of heatiness with the four indices. (Of the cases, the data of 7 are missing.)

Correlation of the Diagnostic Score of heatiness
with the Four Observation Indices

| | N | P | R |
|---|---|---|---|
| Score values Vs SFR | 174 | 0.973 | — |
| Score values Vs LYZ | 174 | 0.592 | — |
| Score values Vs AMS | 174 | 0.121 | — |
| Score values Vs S-IgA | 174 | 0.000* | −0.428 |

Note:
P < 0.05 signifies significant correlation R expresses the correlation coefficient.

From the above analysis, it is clear that the changes of S-IgA are in negative correlation with the severity of heatiness. The more severe the heatiness, the lower the value of S-IgA.

Self-Control Comparison of the Various Parameters
of the Heaty Syndrome in Different Areas

| | Healthy Vs. | Healthy Vs. | Healthy Vs. | Healthy Vs. | Healthy Vs. |
|---|---|---|---|---|---|
| | Heatiness | Heatiness | Heatiness | Heatiness | Heatiness |
| | (G)*1 | (He)*2 | (G & He) | (G & Hu)*3 | (3 Areas) |
| SFR | No significant | (p < 0.05) | (p < 0.05) | No significant | No significant |
| | difference | | | difference | difference |
| | (p > 0.05) | | | (p > 0.05) | (p > 0.05) |
| LYZ | (p < 0.05) | No significant | (p < 0.05) | No significant | No significant |
| | | difference | | difference | difference |
| | | (p > 0.05) | | (p > 0.05) | (p > 0.05) |
| AMS | (p < 0.05) | (p < 0.05) | No significant | (p < 0.05) | No significant |
| | | | difference | | difference |
| | | | (p > 0.05) | | (p > 0.05) |
| S-IgA | (p < 0.05) | (p < 0.05) | (p < 0.05) | (p < 0.05) | (p < 0.05) |

*1Guangzhou,
*2Henan,
*3(Hu) Hunan

Analysis: From the above table, it is seen that in the three areas heaty state differed significantly from the healthy state in S-IgA. As regards other indices, these areas differed significantly or not significantly with no law to follow.

Correlation of the Various Indices with Heatiness Score Values Obtained on the Basis of Guangdzhau Diagnostic Score Table

TABLE 6
Correlation of Various Test Indices with Heatiness

| | | | Cases in G | | Cases in 3 |
|---|---|---|---|---|---|
| | Cases in G | Cases in He | & He | Cases in Hu | Areas |
| Heatiness | Significant | Significant | Significant | Significant | Significant |
| vs S-IgA | correlation | correlation | correlation | correlation | correlation |
| | (r = −0.428, | (r = −0.324, | (r = −0.382, | (r = −0.317, | (r = −0.349, |
| | p = 0.000) | p = 0.000) | p = 0.000) | p = 0.000) | p = 0.000) |
| Heatiness | No significant | Significant | Significant | No significant | No significant |
| vs AMS | correlation | correlation | correlation | correlation | correlation |
| | | (r = −0.222, | (r = −0.150, | | |
| | | p = 0.000) | p = 0.003) | | |
| Heatiness | No significant | Significant | No significant | No significant | No significant |
| vs LYZ | correlation | correlation | correlation | correlation | correlation |
| | | (r = 0.223, | | | |
| | | p = 0.000) | | | |
| Heatiness | No significant | Significant | Significant | No significant | No significant |
| vs SFR | correlation | correlation | correlation | correlation | correlation |
| | | (r = −0.505, | (r = −0.234, | | |
| | | p = 0.000) | p = 0.000) | | |

In the retrospective and the prospective inspection, the Guangzhou diagnostic score table displays the highest specificity and sensitivity. Based on this table, heatiness score values of the cases collected in the three areas and the correlation of the various indices in inspection demonstrate that the heatiness score value and salivary S-IgA correlate consistently; only S-IgA out of the four indices measures showed significant negative correlation with heatiness at the three sites. This suggests salivary S-IgA can be used as a clinical parameter to assess heatiness and measure the effectiveness of antiheatiness treatments.

The gender-related differences of the various indices varied from area to area, but as to S-IgA, the difference is not significant, suggesting that in examination, there is no need to take gender into consideration concerning S-IgA.

The seasonal differences among patients in the three areas are statistically analyzed. Although the seasons spring, summer, autumn, and winter here are the hallmarks but comparison is based on the 24 solar terms in the calendar for the sake of accuracy.

Seasonal Difference of the Various Test Indices

| Indices | Sp-Su | Sp-Au | Sp-Win | Su-Win | Su-Au | Au-Win |
|---|---|---|---|---|---|---|
| SFR | Yes | Yes | No | No | Yes | Yes |
| Lyz | Yes | Yes | Yes | No | Yes | Yes |
| AMS | No | Yes | No | No | Yes | Yes |
| S-IgA | Yes | Yes | Yes | Yes | Yes | Yes |

Comparison of different season pairs, SFR, LYZ, and AMS presents varied differences, but S-IgA is different in different seasons suggesting that basic immunoglobulin changes with seasons, and when S-IgA is used as an index for diagnosis of heatiness, the seasonal factor should be taken into account.

Comparison of Heatiness Quantitative Indices in Different Areas

| | G vs He | G vs Hu | Hu vs He |
|---|---|---|---|
| SFR | Significant | Significant | Significant |
| | difference | difference | difference |
| | p = 0.001 | p = 0.000 | p = 0.000 |
| LYZ | Significant | No significant | Significant |
| | difference | difference | difference |
| | p = 0.000 | p = 0.083 | p = 0.000 |
| AMS | Significant | No significant | Significant |
| | difference | difference | difference |
| | p = 0.000 | p = 0.079 | p = 0.000 |
| S-IgA | Significant | Significant | No significant |
| | difference | difference | difference |
| | p = 0.000 | p = 0.000 | p = 0.116 |

LYZ, AMS, and S-IgA presented irregular difference in different areas, but SFR is different in different areas.

Heatiness vs. SFR: Clinically, oral dryness exists in most cases of heatiness. It is the direct expression of that which leads to a decline in SFR. This part of the study has shown that seasons have significant influence on SFR, which is different in different seasons. Therefore, salivary secretion should be taken as a dynamic process. There are multiple factors, however, that influence the salivary flow rate. 1) General moisture condition of the body: with 8% loss of water of the body, the salivary flow rate may dwindle to zero. The salivary flow rate, however, will rise with the increase in the body moisture. 2) Body posture & lighting condition: The salivary flow rate is higher in the standing position than the sitting position while it is higher is either posture than in a lying posture. It declines by 30%-40% in darkness. 3) Physiological rhythm: The peak of the salivary flow rate is in the afternoon and evening. At night it decreases to nearly zero in sleep. The secretion of the parotid gland is the highest in winter and declines in summer. 4) Stimulation to the senses: Imagination or catching sight of food brings the maximal influence on the salivary flow rate. 5) Drugs: Many sorts of drugs like antidepressant and drugs for Parkinson syndrome have action on the salivary gland, causing the decline of salivary flow rate.

At the preliminary stage of this study, sampling is conducted with the subjects in the sitting position. Saliva specimens are drawn at a regular interval, in an unstimulated condition, with drug influence expelled, so as to have control of the study deviation. From the results of the study, the seasons, too, are an important factor that influences SFR.

It is found in the study that SFR showed no significant difference in gender. It did not show difference either, before and after recovery of the heaty syndrome. This is possible because there are multiple factors that can influence SFR, and this study failed to put many of them under control. Another possibility is that SFR is not necessarily in correlation with the heaty syndrome, and this may be concluded from the correlation inspection of SFR with the diagnostic score values of heatiness.

Heatiness vs. LYZ: LYZ is known to play a role in the process of generation of oral mucositis. In the RAU patients the activity of salivary LYZ is lower either before or after recovery of the disease than in the normal cases. The difference, however, is markedly shown before treatment of the illness only. This fact implies that the decline of the activity of salivary LYZ in RAU patients significantly reduces the oral natural defense. The activity of LYZ is significantly lower in the recurrent aphtha patients than the normal people, and it rises with the recovery of the ailment. In this part of the study, it is also found that in the heaty patients the activity of LYZ is significantly lower before recovery of the ailment than after it. This demonstrates that LYZ probably has a protective function for the body.

Heatiness vs. AMS: In the 1990s, AMS was a focus of medical research. Under the basic conditions, the spleen deficiency syndrome patients have higher than normal activity of salivary AMS. The activity of AMS shows no difference in gender but it changes with seasons, suggesting that seasons have an important influence on the correlation of AMS. We have found that AMS rises significantly in heatiness. Compared with the healthy state, its rise is significant. The mechanism of its change is yet to be probed into.

Heatiness vs. S-IgA: In the oral immune defense system, S-IgA plays an important part, regarded as an important anti-infectious and anti-allergic barrier of the body. In this study it is found that in heatiness, S-IgA declines significantly and it is significantly lower than in the healthy state, suggesting that it is a physiological protective mechanism. In the study of the correlation of S-IgA with the severity of the heaty syndrome, it is found that the severity of the syndrome has a negative correlation with S-IgA, i.e., the more severe the heatiness the lower the activity of S-IgA. It is also found that gender has no significant influence on S-IgA. In comparison of spring with summer, spring with autumn, the activity of S-IgA shows a significant difference. Seasons are an important factor that influences the activity of S-IgA. The difference between the sexes in S-IgA is examined but no significant difference is found.

### Example 2

### Treatment of Heatiness using Toothpaste

Random double blind studies are conducted wherein patients diagnosed with heatiness in accordance with Example 1 are administered different toothpastes:
Three day study: Dentifrice formulation containing 1.1% Sodium Monofluorophosphate, 1.5 PVM/MA Copolymer (Gantrez), 1.3% pyrophosphate, 2% Zinc citrate trihydrate in a silica base (Colgate 360 Whole Mouth Health-Gum Health Toothpaste) vs. Control 1, a commercial toothpaste containing herbs claiming help to relieve gum problems (such as bleeding, gum pain) etc. in silica base with wintergreen flavor)
Seven day study: Dentifrice formulation containing 0.3% Triclosan,2% PVM/MA Copolymer (Gantrez) and 0.32% sodium fluoride in a silica base (Colgate Total® Triclosan/Gantrez Toothpaste) vs. Dentifrice formulation containing 1.1% Sodium Monofluorophosphate, 1.5% PVM/MA Copolymer (Gantrez), 1.3% pyrophosphate, 2% Zinc citrate trihydrate in a silica base (Colgate 360 Whole Mouth Health-Gum Health Toothpaste) vs. Control 2, a commercial toothpaste containing 1.1% Sodium Monofluorophosphate in a calcium carbonate base with mint flavor”

After 3-day usage, people who use Colgate 360 Whole Mouth Health-Gum Health Toothpaste with 2% Zinc citrate statistically show overall greater reduction in heatiness diagnostic score (DS) reduction compared to those who use control 1. On the 7-day usage, we use LPP Whitening Toothpaste as control. People who use Colgate 360 Whole Mouth Health-Gum Health Toothpaste with 2% Zinc citrate statistically showed overall greater reduction in heatiness diagnostic score, while Colgate Total® Triclosan/Gantrez Toothpaste formula showed even more improvement:

Three Day Trial

| | | | | Comparison versus | |
|---|---|---|---|---|---|
| | | | | Control 1 | |
| | | Baseline | Three-Day | Percent | |
| TREATMENT | n | DS* | DS* | Difference | Sig. |
| 360 WMH | 30 | 189.2 ± 50.3 | 153.8 ± 48.0 | −10.6% | P < 0.05 |
| Gum Health | | | | | |
| Toothpaste | | | | | |
| Control 1 | 30 | 187.8 ± 37.5 | 172.6 ± 40.9 | | |

(*Diagnostic Score expressed as mean score +/− standard deviation)

Seven day trial

| | | | | Comparison versus | |
|---|---|---|---|---|---|
| | | | | Control 2 | |
| | | Baseline | Seven-Day | Percent | |
| TREATMENT | n | DS* | DS* | Difference | Sig. |
| 360 WMH | 30 | 189.2 ± 50.3 | 115.1 ± 61.6 | −16.2% | P < 0.05 |
| Gum Health | | | | | |
| Toothpaste | | | | | |
| Triclosan/ | 30 | 175.0 ± 45.1 | 97.6 ± 57.1 | −21.3% | P < 0.05 |
| Gantrez | | | | | |
| Toothpaste | | | | | |
| Control 2 | 30 | 178.3 ± 32.1 | 137.4 ± 46.3 | | |

(*Diagnostic Score expressed as mean score +/− standard deviation)

1. A method of assessing the severity of heatiness in a patient comprising measuring the patient's level of salivary S-IgA, wherein a lower level of S-IgA compared to a control, normal or baseline value indicates a more severe heatiness condition.

2. The method of claim 1 further comprising assessing a patient by checking for one or more of the symptoms listed in Table A:
TABLE A

| Symptoms | value | Symptoms | value | Symptoms | value |
|---|---|---|---|---|---|
| Mouth ulcer | ***23 | Ocular | *8 | Low-grade | **15 |
| | | itching | | fever | |
| Dry mouth | **14 | Secretion of | **18 | Insomnia | *8 |
| | | the eye | | | |
| Bitter taste | **15 | Tinnitus | **11 | Tantrum | **17 |
| of mouth | | (ear ringing) | | | |
| Halitosis | **17 | Dry pharynx | **13 | Yellow urine | *9 |
| Gum swelling, | **17 | Sore throat | **15 | Constipation | *8 |
| pain or | | | | | |
| bleeding | | | | | |
| Red tongue | *9 | Acne | *6 | Stuffiness | *6 |
| Dryness in the | **15 | Dizziness | *6 | Yellow musci | **15 |
| nasal cavity | | | | | |
| Nose bleeding | *10 | Scurf | *6 | Frequent and | **11 |
| | | (scales, flakes) | | weak pulse | |
| Dryness of | *9 | Dryness-heat | **17 | Frequent and | **13 |
| the eyes | | | | strong pulse | |

and assigning a score to the patient based on the symptoms exhibited and the values assigned in Table A, wherein a higher score indicates a more severe heatiness condition.

3. The method in accordance with claim 1 wherein the salivary S-IgA level is measured using antibodies to S-IgA.

4. The method in accordance with claim 1 wherein the patient has not had any oral intake for at least one hour prior to the measurement of the salivary S-IgA.

5. The method of claim 1 wherein the normal or baseline level of S-IgA is calculated taking into consideration the season of the year.

6. A computer-assisted system for diagnosis of heatiness, wherein a user enters data regarding the S-IgA levels, presence or absence of symptoms as listed in Table A via a website, the data is uploaded into a calculating program to permit calculation of a heatiness diagnostic score, the heatiness diagnostic score is then displayed to the user, and based on the heatiness diagnostic score, information regarding heatiness and/or appropriate methods of treatment is provided to the user.

7. A method of treatment comprising diagnosing a patient as suffering from heatiness in accordance with any of the foregoing claims, and treating the patient by administering an oral care product comprising an effective amount of an antiheatiness agent.

8. The method of claim 7 wherein the antiheatiness agent is one or more anti-heatiness agents selected from: berberine and jateorhizine from rhizome of Chinese goldthread, baicalin from root of Baikal skullcap, matrine and oxymatrine from root of lightyellow sophora, andrographolide from common andrographis herb, houttuynine sodium bisulfite from herb of heartleaf houttuynia, mangiferin from rhizome of anemarrhena, and alkanin from redroot gromwell; chrysanthemum lactone from flos chrysanthemi indici, chlorogenic acid from honeysuckle flower, tea polyphenols from tea leaf, and magnolol from bark of magnolia.

9. The method of claim 7 wherein the antiheatiness agent comprises a synthetic antibacterial or anti-inflammatory agent.

10. The method of claim 9 wherein the antiheatiness agent is selected from triclosan, zinc citrate, zinc oxide, zinc lactate and combinations thereof.

11. The method of claim 7 wherein the oral care product is a toothpaste.

12. The method of claim 7 wherein the oral care product is administered to the oral cavity of the patient on at least a daily basis for a period of at least one week or until symptoms subside.

13. An oral care product comprising an effective amount of an antiheatiness agent, for use in a method of treating heatiness according to claim 7.

14. The oral care product of claim 13 wherein the antiheatiness agent is one or more anti-heatiness agents selected from: berberine and jateorhizine from rhizome of Chinese goldthread, baicalin from root of Baikal skullcap, matrine and oxymatrine from root of lightyellow sophora, andrographolide from common andrographis herb, houttuynine sodium bisulfite from herb of heartleaf houttuynia, mangiferin from rhizome of anemarrhena, and alkanin from redroot gromwell; chrysanthemum lactone from flos chrysanthemi indici, chlorogenic acid from honeysuckle flower, tea polyphenols from tea leaf, and magnolol from bark of magnolia.

15. The oral care product of claim 13 wherein the antiheatiness agent comprises a synthetic antibacterial or anti-inflammatory agent.

16. The oral care product of claim 15 wherein the antiheatiness agent is selected from triclosan, zinc citrate, zinc oxide, zinc lactate, and combinations thereof.

17. The oral care product of claim 16 wherein the oral care product is selected from
a dentifrice comprising 0.3% triclosan, 2% methyl vinyl ether/maleic anhydride copolymer and 0.32% sodium fluoride in a silica base and
a dentifrice comprising 1.1% sodium monofluorophosphate, 1.5% methyl vinyl ether/maleic anhydride copolymer, 1.3% pyrophosphate, and 2% zinc citrate trihydrate in a silica base.

18. Use of an oral care product comprising an effective amount of an antiheatiness agent in the manufacture of a medicament for the treatment of heatiness according to claim 7.

19. Use of an oral care product according to claim 14 in the manufacture for the treatment of heatiness.
